(19) United States
(12) Patent Application Publication
(10) Pub. No.: US 2015/0003582 A1
(43) Pub. Date: Jan. 1, 2015

(54) Apparatus for Measuring the Composition of a Multi-Phase Mixture Flow

(71) Applicant: SIEMENS AKTIENGESELLSCHAFT, Muenchen (DE)

(72) Inventors: Stepan Polikhov, Ramenskoye (RU); Reiner Franz Schulz, Erlangen (DE)

(21) Appl. No.: 14/345,882

(22) PCT Filed: Sep. 20, 2011
(86) PCT No.: PCT/RU2011/000716

§ 371 Date: Aug. 20, 2014

(51) Int. Cl. G01N 23/12; G01N 23/087; G01N 33/28

(52) U.S. Cl. CPC G01N 23/12; G01N 33/2823; G01N 23/087
USPC 378/53

(57) ABSTRACT

An apparatus for measuring a composition of a multi-phase mixture, the multi-phase mixture comprising at least one liquid phase and at least one gaseous phase, comprises: a measurement tube that forms a conduit configured for receiving a flow of the multi-phase mixture; a radiation part configured for irradiating the multi-phase mixture in the measurement tube with electromagnetic radiation; a detector configured for detecting radiation that passes through the multi-phase mixture in the measurement tube; and an analyzer configured for determining the composition of the multi-phase mixture based on the detected radiation and calibration data of the at least one liquid phase and the at least one gaseous phase. A data acquisition part is configured for acquiring calibration data from radiation detected by the detector that passes through a calibration vessel filled with the multi-phase mixture or respective phases of the multi-phase mixture from the measurement tube.

### RELATED APPLICATIONS

This application is the National Stage of International Application No. PCT/RU2011/000716, filed Sep. 20, 2011, the entire contents of which are hereby incorporated herein by reference.

### TECHNICAL FIELD

The present teachings relate generally to multi-phase mixture flows and to measuring the compositions thereof.

### BACKGROUND

In many industries, techniques for measuring the composition of a multi-phase mixture flow are used. For example, in the gas and oil industry, non-intrusive methods for measuring the multi-phase mixture flow emanating from an oil or gas well are used.

U.S. Pat. No. 6,097,786 describes a method and apparatus for determining the composition of a multi-phase mixture flow based on X-ray radiation. In this method, the multi-phase mixture flow is irradiated with high-energy and low-energy X-ray radiation. The radiation that passes through the flow is measured by a multi-layer detector. Since the attenuation of the radiation depends on the composition of the multi-phase mixture flow, the fractions of the different phases may be determined.

International Patent Document No. WO 2011/005133 A1 describes an apparatus and method for measuring the flow velocity of a multi-phase fluid mixture. In this method, images of the spatial distributions of the photons from X-ray sources are detected at different intervals of time. Based on those distributions, the flow rate of the fluid mixture may be determined.

In conventional analyses of a multi-phase mixture flow, the measurement apparatus is calibrated manually. The different absorption coefficients of the phases in the flow are determined in advance so that the composition of the flow may be calculated based on those absorption coefficients. Manual calibration is time-consuming and involves sampling of the flow at the location of the apparatus by an operator. As a result, manual calibration is not repeated very often and may lead to inaccurate measurements of the composition of the multi-phase mixture flow.

### SUMMARY AND DESCRIPTION

The scope of the present invention is defined solely by the appended claims, and is not affected to any degree by the statements within this summary.

The present embodiments may obviate one or more of the drawbacks or limitations in the related art. For example, in some embodiments, an apparatus and a method for measuring the composition of a multi-phase mixture flow using self-calibration are provided.

An apparatus in accordance with the present teachings measures the composition of a multi-phase mixture flow including at least one liquid phase (e.g., different liquids, such as oil and water) and at least one gaseous phase (e.g., different kinds of gases). In some embodiments, the apparatus is configured for measuring a flow of liquid and gaseous hydrocarbons emanating from a well. The apparatus includes a measurement tube that forms a conduit for a flow of a multi-phase mixture. The term “measurement tube” is to be interpreted broadly and may refer to any measurement section having an arbitrary cross-section (e.g., a rectangular or a circular cross-section). The apparatus further includes a radiation part configured for irradiating the multi-phase mixture in the measurement tube with electromagnetic radiation. A detector is provided for detecting the radiation of the radiation part that passes through the multi-phase mixture in the measurement tube. An analyzer is used for determining the composition of the multi-phase mixture based on the detected radiation and calibration data of the at least one liquid phase and the at least one gaseous phase. The measurement principle used in accordance with the present teachings may be based on conventional methods. For example, the measurement may be based on the method described in U.S. Pat. No. 6,097,786 wherein radiation at different energy levels is detected in order to determine the composition of a multi-phase mixture flow. The entire contents of U.S. Pat. No. 6,097,786 are hereby incorporated by reference.

Apparatuses in accordance with the present teachings implement a self-calibration unit. The unit includes a calibration vessel that is arranged adjacent to the measurement tube. During operation of the apparatus, the radiation part may irradiate the calibration vessel and the detector may detect radiation of the radiation part that passes through the calibration vessel.

The calibration vessel is connectable to the measurement tube, such that the calibration vessel is filled with the multi-phase mixture or respective phases of the multi-phase mixture from the measurement tube. The calibration unit further includes a data acquisition part configured for acquiring calibration data from radiation detected by the detector that passes through the calibration vessel when the calibration vessel is filled with the multi-phase mixture or respective phases of the multi-phase mixture from the measurement tube.

In some embodiments, automatic calibration may be performed by filling a calibration vessel with a multi-phase mixture and automatically acquiring calibration data via the data acquisition part. Thus, manual calibration may be avoided. The automatic calibration may be performed at regular intervals to provide accurate measurements of the composition of a multi-phase mixture flow.

In some embodiments, the radiation from the radiation part includes high-energy electromagnetic radiation with a photon energy of at least 10 KeV. X-ray radiation and/or gamma radiation may be used since the radiation is only partially absorbed by the multi-phase mixture and the radiation may be detected by the detector.

As described above, conventional methods may be used for determining the composition of the multi-phase mixture flow (e.g., the method described in U.S. Pat. No. 6,097,786). In such methods, the radiation part may generate at least two different radiation pulses. The at least two different radiation pulses may include a first pulse having a low energy level and a second pulse having a high energy level. The detector may detect the different radiation pulses. In addition, the analyzer is configured for determining the composition of the multi-phase mixture flow based on the detected different radiation pulses and the calibration data. The calibration data is acquired by the data acquisition part from the different radiation pulses detected by the detector that pass through the calibration vessel when the calibration vessel is filled with the multi-phase mixture or respective phases of the multi-phase mixture from the measurement tube.

In some embodiments, the calibration data includes absorption coefficients for the phases of the multi-phase mixture with respect to the radiation of the radiation part. The absorption coefficients may be used to calculate the different fractions of the phases of the multi-phase mixture flow in the measurement tube.

To facilitate calibration, the measurement conditions for the measurement tube and for the calibration vessel may be similar. In some embodiments, the measurement tube and the calibration vessel are made of the same material and/or have the same cross-section. For example, the measurement tube and/or the calibration vessel may be from materials that are transmissive to electromagnetic radiation (e.g., high-energy electromagnetic radiation). These materials include beryllium bronze and/or carbon fiber and/or glassy carbon.

Furthermore, in some embodiments, the measurement tube and/or the calibration vessel may have an elliptical cross-section or a cross-section in the form of an elongated hole. An elliptical cross-section or a cross-section in the form of an elongated hole provides high resistance with respect to the pressure in the tube and the vessel. An elliptical cross-section or a cross-section in the form of an elongated hole also prevents the paths of the radiation beams through the tube and the vessel from varying too much.

In some embodiments, the measurement tube and the calibration vessel are arranged symmetrically with respect to the radiation part and the detector. The radiation reaching the detector that has passed through the measurement tube and the calibration vessel, respectively, does not change when the positions of the measurement tube and the calibration vessel are reversed. Since the measurement conditions for the tube and the vessel are substantially the same, a straightforward calculation based on the calibration data without any conversions may be performed to determine the composition of a multi-phase mixture flow.

In order to provide similar conditions during calibration and measurement, the temperatures in the calibration vessel and the measurement tube may be the same. In some embodiments, a section of the measurement tube and a section of the calibration vessel are in direct contact with each other to provide a good thermal transfer. Furthermore, the measurement tube and the calibration vessel may be surrounded by a thermal insulation such that the thermal conditions of the tube and the vessel are not affected by the environment.

A detector for use in accordance with the present teachings may be implemented in various ways. For example, the detector may include one or more detection sensors. In some embodiments, the detector includes a matrix detector configured to provide spatial resolution of the detected radiation. The detector may also or alternatively include two detection sensors, wherein a first detection sensor is configured for detecting radiation that passes through the measurement tube and the second detection sensor is configured for detecting radiation that passes through the calibration vessel. By using a matrix detector, different phases in the multi-phase mixture in the calibration vessel may be distinguished after segregation.

In some embodiments, the measurement tube and the calibration vessel extend in the vertical direction during operation of the apparatus, thereby facilitating a gravitational stratification of the different phases in the multi-phase mixture inside the calibration vessel.

In some embodiments, in order to connect the calibration vessel with a measurement tube, a valve system is provided. The valve system includes one or more valves and one or more conduits. The valve system is arranged between the measurement tube and the calibration vessel and is controllable by the data acquisition part. The valve system may be used to fill the calibration vessel with the multi-phase mixture or respective phases of the multi-phase mixture from the measurement tube.

In some embodiments, the valve system includes a sampling probe positioned in the measurement tube and connected with the calibration vessel via a first conduit that includes a first valve. The calibration vessel is filled with the multi-phase mixture from the measurement tube when the first valve is opened.

Furthermore, the valve system may include a second conduit that includes a second valve. The at least one gaseous phase of the multi-phase mixture may be interchanged between the measurement tube and the calibration vessel when the second valve is opened.

The valve system may also include a third conduit that includes a third valve. The multi-phase mixture in the calibration vessel is fed back to the measurement tube when the third valve is opened. The third conduit may be connected to a flow restriction in the measurement tube. A reduction in pressure in the measurement tube due to the flow restriction results in a good return flow to the measurement tube.

In some embodiments, the valve system includes a fourth conduit that includes a fourth valve. The at least one gaseous phase of the multi-phase mixture in the calibration vessel is released from the calibration vessel when the fourth valve is opened. Such embodiments may be used to measure the composition of stable and unstable gas condensates in a gas condensate flow.

In addition to the above-described measurement of the composition of a multi-phase mixture, an apparatus in accordance with the present teachings may also be used for the measurement of flow rate based on images taken by the detector. In some embodiments, the method described in International Patent Publication No. WO 2011/005133 A1 may be used to determine the flow rate. The entire contents of WO 2011/005133 A1 are hereby incorporated by reference.

In addition to the above-described apparatus, the present teachings also provide a method for calibrating the apparatus. The method includes: (i) filling the calibration vessel with the multi-phase mixture of respective phases from the measurement tube; (ii) irradiating the calibration vessel with electromagnetic radiation from the radiation part and detecting the radiation that passes through the calibration vessel by the detector; and (iii) acquiring calibration data by the data acquisition part from the radiation detected by the detector.

In some embodiments, the calibration vessel is successively filled with the at least one gaseous phase and the at least one liquid phase of the multi-phase mixture from the measurement tube. In other embodiments, the calibration vessel is filled in a reverse order. The acts of (ii) irradiating and (iii) acquiring are performed for both the at least one liquid phase and the at least one gaseous phase. The method may be used for a gas condensate flow.

In some embodiments, the calibration vessel is not filled separately with the different phases. Instead, the calibration vessel is filled with the multi-phase mixture from the measurement tube whereupon stratification of the multi-phase mixture takes place. In the stratification, the at least one liquid phase and the at least one gaseous phase are separated in the vessel. The acts of (ii) irradiating and (iii) acquiring are performed after the stratification. The method may be used for a multi-phase mixture flow emanating from an oil well (e.g., that may include oil, water, and gas).

In some embodiments, the at least one liquid phase is separated into different kinds of liquids (e.g., oil and water) during stratification, and calibration data are acquired for each kind of liquid.

In some embodiments, the multi-phase mixture is a gas condensate, wherein the ratio of stable and unstable condensates at atmospheric pressure is determined by the data acquisition part.

### BRIEF DESCRIPTION OF THE DRAWINGS

FIG. 1 shows a front view of an exemplary multi-phase flow meter in accordance with the present teachings in partial cross-section.

FIG. 2 shows a top view of the exemplary flow meter of FIG. 1.

FIG. 3 shows a view from the left of the exemplary flow meter of FIG. 1.

FIG. 4 shows a view from the right of the exemplary flow meter of FIG. 1.

### DETAILED DESCRIPTION

An exemplary flow meter that may be used at oil wells or gas wells to determine the composition of a multi-phase mixture flow emanating from the well is described. The flow meter may be installed in oil pipelines or gas pipelines to analyze the flow therein. In addition to determining the composition of the multi-phase flow, the flow meter described below may optionally also be used to determine the flow velocity based on the method described in document WO 2011/005133 A1. However, in some embodiments, the flow meter includes only the function of measuring the composition of the multi-phase mixture flow.

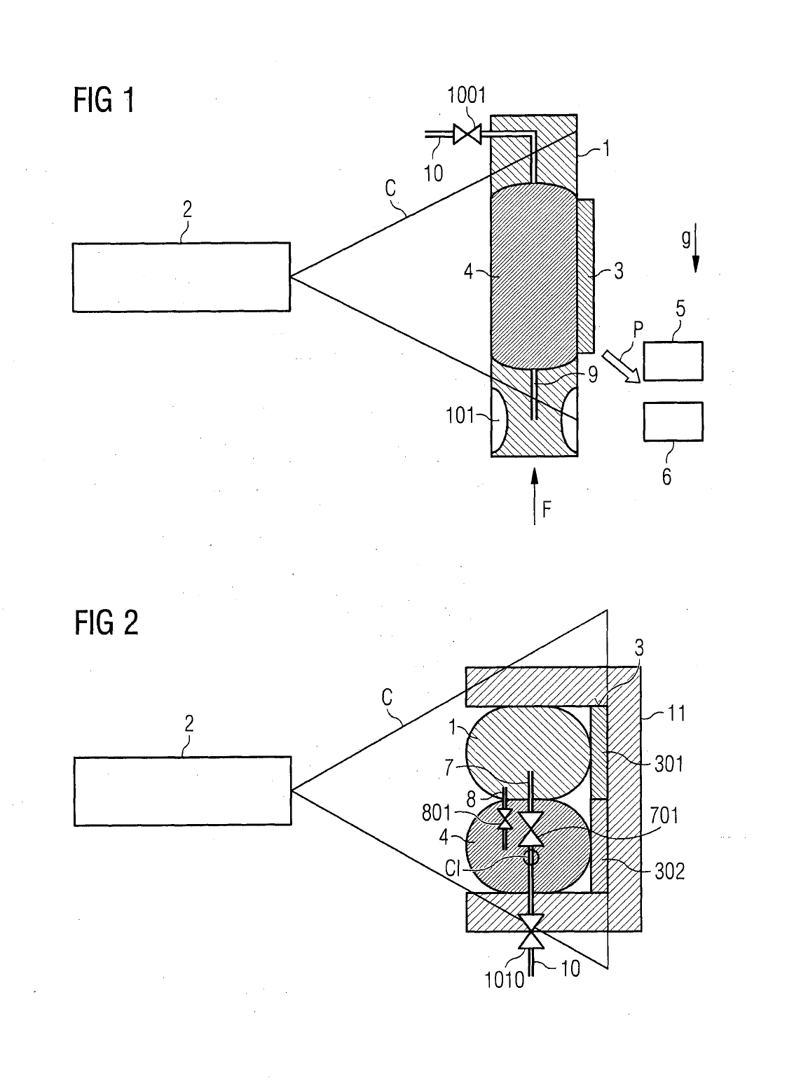

FIG. 1 shows a front view of the flow meter. The meter includes a measurement tube 1 that is arranged in a vertical direction with respect to the force of gravity. The force of gravity is indicated by arrow g. In the measurement tube 1, a multi-phase flow is conveyed upwards in the vertical direction. The direction of the flow is indicated by the arrow F. In order to analyze the phases included in the multi-phase flow, a radiation part 2 in the form of an X-ray source irradiates the measurement tube 1 with an X-ray beam. The cross-section of the X-ray beam is indicated by the triangle C. The material of the measurement tube 1 is transparent for X-ray beams. In some embodiments, the measurement tube 1 is made of beryllium bronze, carbon fiber, glassy carbon, or any other material that is relatively transparent to X-ray radiation. A part of the radiation is absorbed by the multi-phase mixture inside the measurement tube 1. The rest of the radiation (e.g., the non-absorbed radiation) is detected by a detector 3 behind the measurement tube 1. The detector 3 includes matrix sensors configured to provide a spatial resolution of the detected intensity in the vertical and the horizontal direction.

In some embodiments, as shown in FIG. 1, the measurement tube 1 is irradiated with radiation on a high energy level and a low energy level. The photons on the high energy level have an absorption coefficient that is the same for both oil and water in the multi-phase flow of the well. By contrast, the photons on the low energy level are absorbed substantially more strongly by water than by oil. With respect to the gases in the multi-phase mixture, the absorption coefficients of all gases are low for both levels of energy. The detector 3 may resolve the beams with the different energy levels. By using the above-described relationship between the energy levels of the photons and the absorption coefficients of the photons in the multi-phase flow, the composition of the multi-phase flow may be determined (e.g., the percentages of the different materials in the flow may be determined). A technique that may be used to determine the composition is described in U.S. Pat. No. 6,265,713 B1.

The calculation of the composition of the multi-phase mixture flow uses the absorption coefficients for the gaseous phase and the liquid phase (in some embodiments, separate coefficients for the oil phase, the water phase, and the gas phase or phases). The coefficients are calibration parameters pre-determined in a calibration process. Conventionally, the flow meter is calibrated manually (e.g., a sample is taken from the multi-phase mixture flow and analyzed in a separate process). This manual calibration is time-consuming and involves a person visiting the well or the pipeline to take a sample. By contrast, the flow meter shown in FIG. 1 may be used for an automatic calibration (e.g., at regular intervals), and may thereby avoiding disadvantages of manual calibration.

To perform automatic calibration, the flow meter shown in FIG. 1 includes a calibration vessel 4 that is arranged adjacent to the measurement tube 1. In the view in FIG. 1, the calibration vessel 4 is arranged in front of the measurement tube 1. The calibration vessel 4 is also irradiated by the beam C of the X-ray source 2. The detector 3 extends behind the calibration vessel 4 so that the intensity of photons passing through the calibration vessel 4 may also be detected by the detector 3. As further described below, the calibration vessel 4 is connected with the measurement tube 1 by a valve system that includes valves and conduits. In some embodiments, the calibration vessel 4 may be filled with the multi-phase mixture from the measurement tube 1 by opening corresponding valves that are not shown by the view in FIG. 1.

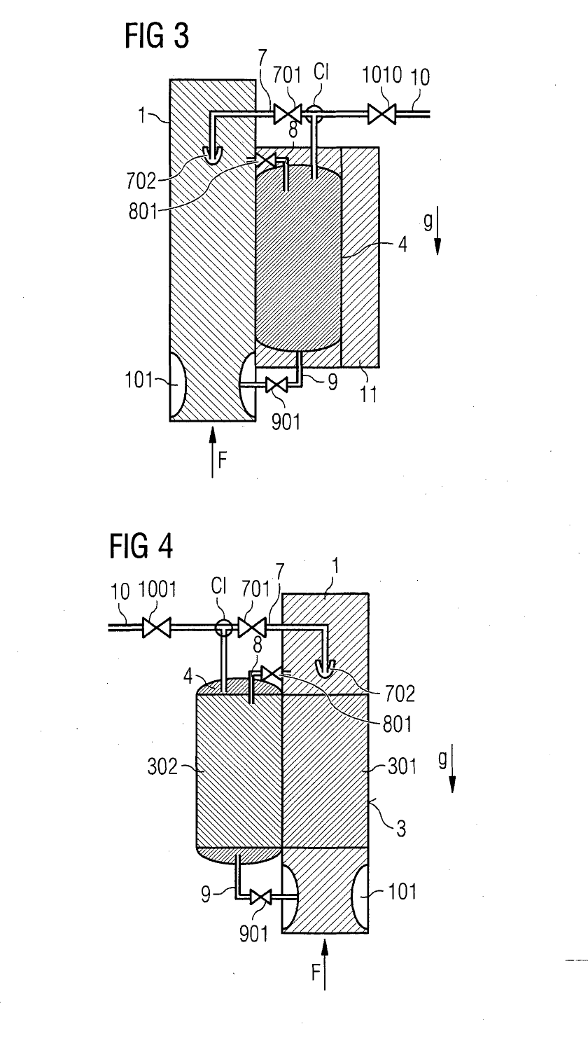

FIG. 1 shows an upper conduit 10 with a valve 1001. The upper conduit 10 is connected to the upper side of the calibration vessel 4. By opening the valve 1001, gases emanating from a multi-phase mixture inside the calibration vessel 4 may exit to the outside atmosphere. Furthermore, a lower conduit 9 is connected with the bottom side of the calibration vessel 4. The lower conduit 9 includes a valve 901 as shown in FIG. 3. By opening the valve 901, the multi-phase mixture flow may be fed back from the calibration vessel 4 to the measurement tube 1. The measurement tube 1 includes in the lower part a flow restriction in the form of a ring-shaped recess 101 that extends around the circumference of the tube 1. The conduit 9 is connected to the measurement tube at the flow restriction 101. Due to the Venturi effect, the pressure at the flow restriction 101 is reduced. As a result, the multi-phase mixture in the calibration vessel 4 is blown down into the measurement tube 1.

To calibrate the flow meter, the calibration vessel 4 is filled via the valve system with multi-phase mixture from the measurement tube 1. Upon filling, gravitational stratification takes place thus separating the different phases and the mixture. In other embodiments, the calibration vessel is separately filled with the liquid phases and gaseous phases from the multi-phase flow as further described below. The calibration vessel is then irradiated by the X-ray source 2 with the two levels of energy. The corresponding intensity of the photons passing through the calibration vessel 4 is measured by the detector 3. As the detector 3 is a matrix detector having a spatial resolution, the different phases may be distinguished due to the different absorption behaviors. Eventually, the absorption coefficients for the different phases are determined. These absorption coefficients are used as calibration data for the measurements of the multi-phase mixture flow in the measurement tube.

As indicated by the arrow P in FIG. 1, the data from the detector 3 are processed in an analyzer 5 and a data acquisition part 6 that are not shown in other figures. The analyzer 5 performs the calculations in order to determine the composition of the multi-phase mixture based on the detected intensities of the radiation passing through the tube 1. As describe above, the determination may use the method described in U.S. Pat. No. 6,097,786. For the calculations, the analyzer 5 uses calibration data that are determined by the data acquisition part 6. The data acquisition part 6 obtains the detected photon intensities with respect to the radiation that passes through the calibration vessel. The data acquisition part 6 uses these data to calculate the absorption coefficients of the different phases that are then used by the analyzer 5. The analyzer 5 and the data acquisition part 6 are implemented in hardware and software (e.g., in the form of a computer). The analyzer and the data acquisition part may form software programs installed on the same computer, such that the analyzer and the data acquisition part are integrated into a single unit.

FIG. 2 shows a top view of the flow meter shown in FIG. 1. As shown by FIG. 2, the detector 3 includes first detection sensor 301 and second detection sensor 302. Both of first detection sensor 301 and second detection sensor 302 are matrix detectors. The first detection sensor 301 is configured for detecting radiation passing through measurement tube 1. The second detection sensor 302 is configured for detecting radiation passing through the calibration vessel 4. In order to facilitate calibration, the sensitivities of the first detection sensor 301 and the second detection sensor 302 are the same, and the position of the first detection sensor 301 with respect to the measurement tube 1 corresponds to the position of the second detection sensor 302 with respect to the calibration vessel 4. Furthermore, the cross-section and the size of the measurement tube 1 and the calibration vessel 4 are substantially the same. Moreover, the measurement tube 1 and the calibration vessel 4 are formed of the same material, such that the transmission of the radiation is the same for the tube 1 and the vessel 4.

The choice of the cross-sectional shape of the measurement tube is based on the criteria that the tube withstands high pressure (e.g., optimally a circular cross-section) and that the path variance for different X-ray beams passing through the measurement tube to the detector be minimal (e.g., optimally a square cross-section). Based on these criteria, an elliptic-like cross-section may be used for the shape of the measurement tube 1. For example, the shape of the measurement tube 1 has the form of an elongated hole that includes two flat sections and two circular sections. As shown in FIG. 1, the calibration vessel 4 has the same form as the measurement tube 1 and is arranged adjacent to the measurement tube 1 such that the two flat surfaces of the tube 1 and the vessel 4 are in direct contact.

As described above, both the measurement tube 1 and the calibration vessel 4 may be made of the same or similar material and have identical forms. Furthermore, the temperature of the calibration vessel 4 may be close to the temperature of the measurement tube 1. To fulfill these criteria, a good thermal contact is provided via the contacting flat surfaces of the tube 1 and the vessel 4. Furthermore, to insulate the tube 1 and the vessel 4 from the environment, a thermal insulation 11 (not shown in FIG. 1) is arranged around the tube 1 and the vessel 4.

In addition to the conduit 10 and the valve 1010, other conduits and valves are shown in FIG. 2. As shown in FIG. 3, a conduit 7 extends inside the measurement tube 1 to a sampling probe 702. The conduit 7 includes a valve 701. By opening the valve 701, the multi-phase mixture may flow via the conduit 7 to a junction indicated by circle CI. At this junction, the conduit 7 extends on one side down to the vessel 4 so that the multi-phase mixture may enter the vessel 4. Furthermore, the junction extends to the conduit 10 via valve 1010. By opening the valve 1010, gases of the multi-phase mixture may exit to the outside.

As shown by FIG. 2, a further conduit 8 includes a valve 801. The conduit 8 extends in a vertical direction in the measurement tube 1 and in a horizontal direction in the calibration vessel 4, as shown in FIG. 3. By opening the valve 801, the gaseous phases of the multi-phase mixture in the measurement tube 1 may enter the calibration vessel 4.

FIG. 3 shows a side view from the left of the flow meter of FIG. 1. As shown in FIG. 3, the conduit 7 extends inside the measurement tube 1 and ends at a sampling probe 702. The junction CI between the conduit 10 and the conduit 7 is shown in FIG. 3. The sampling probe 702 facilitates transportation of the multi-phase mixture inside the measurement tube to the calibration vessel 4 when the valve 701 is opened. Furthermore, as shown in FIG. 3, the conduit 8 with the corresponding valve 801 is arranged between the measurement tube 1 and the calibration vessel 4. As further shown in FIG. 3, the conduit 9 includes a valve 901 and ends in the flow restriction 101 as described above.

FIG. 4 shows a view from the right of the flow meter of FIG. 1, and shows the structure of the detector 3. The detector 3 includes two identical matrix detectors. The first matrix detector 301 is arranged adjacent to the measurement tube 1. The second matrix detector 302 is arranged adjacent to the calibration vessel 4.

In some embodiments, the arrangement of the measurement tube 1 with respect to the X-ray source 2 and the detector 301 corresponds to the arrangement of the calibration vessel 4 with respect to the X-ray source 2 and the detector 302. Furthermore, in some embodiments, the material and the size of the vessel 4 and the tube 1 are the same and the same type of detectors 301 and 302 are used. The multi-phase mixtures in the vessel and the tube are in the same thermal condition. As a result, the absorption coefficients calculated by the data acquisition part 6 may be used directly by the analyzer 5 without any conversion calculations. Hence, facile calibration of the flow meter is achieved, and the calibration data quality is improved.

Two operation modes of the flow meter are now described in reference to FIGS. 1-4. In both operation modes, the valves of the valve system are opened and closed in a predetermined manner. The control of the valves is performed by the data acquisition part 6 shown in FIG. 1.

In a first operation mode, a gas condensate flow emanating from a gas well is calibrated. In a gas condensate flow, 90% to 95% by volume of the mixture is gas. To perform calibration of the gaseous phase, the valve 801 of the flow meter is opened, while all of the other valves are closed, such that the calibration vessel is filled with gas mixture. In this condition, the calibration data for the pure gas phase may be acquired using the X-ray source 2 and the detector 3.

The liquid phase of the gas condensate is collected by opening the valves 801 and 701 while all other valves are closed, such that the calibration vessel 4 is filled with the liquid phase. This process may take some time since the fraction of liquid in the gas condensate is rather low by comparison to other multi-phase mixtures. After the collection of the liquid phase, calibration data for this phase are acquired via the X-ray detector 2 and the detector 3.

Optionally, an additional measurement may be performed during calibration. For the optional additional measurement, the valve 6 is opened while all of the other valves are closed, such that atmospheric pressure will settle down in the calibration vessel. As a result, non-stable condensate will evaporate whereas stable fractions will stay in the calibration vessel. The ratio between stable and unstable condensates may be determined via measurement of the liquid level in the calibration vessel (e.g., due to the spatial resolution ability of the matrix detector 302) by comparing the levels in the vessel before and after opening the valve 1010.

Purging of the calibration vessel may be performed. During purging, all valves except the valves 801 and 901 are closed. Since the pressure in the restricted flow area 101 is reduced, the content of the calibration vessel will be blown down into the measurement tube. Thus, the calibration vessel is again filled with the gas fraction of the multi-phase mixture from the measurement tube and the acts described above may be repeated.

In a second operational mode, the calibration procedure is performed for a multi-phase flow emanating from an oil well. Such a multi-phase flow contains water, gas, and oil phases. In this second operational mode, the valve 701 and the valve 901 are opened. As a result, the multi-phase mixture will flow through the calibration vessel. The duration of this act is determined such that the multi-phase mixture in the calibration vessel 4 will be completely exchanged by the mixture from the measurement tube. Since the calibration vessel 4 is being filled from the top and the valve 901 is located at the bottom, the gas content of the mixture will be higher in the calibration vessel as compared to the actual flow in the measurement tube.

Stratification of the mixture in the calibration vessel may be performed. During stratification, only valve 801 is opened. Due to gravitational stratification, segregation of the mixture takes place because of the different densities of oil, water, and gas. The duration of this act may be long enough to allow for complete segregation of the mixture. As a result, the calibration vessel content is distributed such that the vessel contains water at the bottom, oil in the middle, and gas at the top.

Data acquisition takes place with the X-ray source 2 and the detector 3. Since oil, water, and gas have different X-ray absorptions, the different phases may be distinguished by the matrix detector 302. Hence, calibration data in the form of absorption coefficients may be obtained for the oil, water, and gas phases.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding claim—whether independent or dependent—and that such new combinations are to be understood as forming a part of the present specification.

1. An apparatus for measuring a composition of a multi-phase mixture, the multi-phase mixture comprising at least one liquid phase, at least one gaseous phase, or at least one liquid phase and at least one gaseous phase, the apparatus comprising:
a measurement tube that forms a conduit configured for receiving a flow of the multi-phase mixture;
a radiation part configured for irradiating the multi-phase mixture in the measurement tube with electromagnetic radiation;
a detector configured for detecting radiation that passes through the multi-phase mixture in the measurement tube;
an analyzer configured for determining the composition of the multi-phase mixture based on the detected radiation and calibration data of the at least one liquid phase and the at least one gaseous phase;
a calibration vessel configured to be arranged adjacent to the measurement tube, wherein the radiation part is further configured to irradiate the calibration vessel, wherein the detector is further configured to detect radiation that passes through the calibration vessel, and wherein the calibration vessel is configured to be connected to the measurement tube and to be filled with the multi-phase mixture or respective phases of the multi-phase mixture from the measurement tube; and
a data acquisition part configured for acquiring calibration data from radiation detected by the detector that passes through the calibration vessel when the calibration vessel is filled with the multi-phase mixture or the respective phases of the multi-phase mixture from the measurement tube.

2. The apparatus to of claim 1, wherein the radiation from the radiation part comprises a high energy electromagnetic radiation with a photon energy of at least 10 KeV.

3. The apparatus of claim 1, wherein:
the radiation part is further configured to generate at least a first radiation pulse and a second radiation pulse, wherein the first radiation pulse comprises a low energy level and wherein the second radiation pulse comprises a high energy level;
the detector is further configured to detect the first radiation pulse and the second radiation pulse;
the analyzer is further configured for determining the composition of the multi-phase mixture based on the detected first radiation pulse, the detected second radiation pulse, and the calibration data; and
the calibration data is acquired by the data acquisition part from the first radiation pulse and the second radiation pulse detected by the detector.

4. The apparatus of claim 1, wherein the calibration data comprises absorption coefficients for phases of the multi-phase mixture.

5. The apparatus of claim 1, wherein the measurement tube and the calibration vessel are made of the same material, have the same cross-section, or are made of the same material and have the same cross-section.

6. The apparatus of claim 1, wherein the measurement tube, the calibration vessel, or the measurement tube and the calibration vessel are made of beryllium bronze, carbon fiber, glassy carbon, or a combination thereof.

7. The apparatus of claim 1, wherein the measurement tube, the calibration vessel, or the measurement tube and the calibration vessel have a cross-section that is elliptical, in the form of an elongated hole, or elliptical and in the form of an elongated hole.

8. The apparatus of claim 1, wherein the measurement tube and the calibration vessel are arranged symmetrically with respect to the radiation part and the detector, such that radiation reaching the detector that has passed through the measurement tube and the calibration vessel, respectively, does not change upon interchanging positions of the measurement tube and the calibration vessel.

9. The apparatus of claim 1 wherein a section of the measurement tube and a section of the calibration vessel are in direct contact.

10. The apparatus of claim 1 wherein the measurement tube and the calibration vessel are surrounded by a thermal insulation.

11. The apparatus of claim 1, wherein (a) the detector comprises a matrix detector configured for spatial resolution of the detected radiation, (b) the detector comprises a first detection sensor and a second detection sensor, the first detection sensor is being configured for detecting radiation passing through the measurement tube and the second detection sensor being configured for detecting radiation passing through the calibration vessel, or (c) the detector comprises the matric detector, the first detection sensor, and the second detection sensor.

12. The apparatus of claim 1, wherein the measurement tube and the calibration vessel extend in a vertical direction during operation of the apparatus.

13. The apparatus of claim 1, further comprising a valve system, wherein the valve system comprises one or more valves and one or more conduits, wherein the valve system is arranged between the measurement tube and the calibration vessel, and wherein the valve system is controllable by the data acquisition part and is configured for filling the calibration vessel with the multi-phase mixture or respective phases of the multi-phase mixture from the measurement tube.

14. The apparatus of claim 13, wherein the valve system further comprises a sampling probe positioned in the measurement tube, wherein the sampling probe is connected via a first conduit with the calibration vessel, wherein the first conduit comprises a first valve, and wherein the calibration vessel is filled with the multi-phase mixture from the measurement tube when the first valve is opened.

15. The apparatus of claim 13, wherein the valve system further comprises a second conduit, wherein the second conduit comprises a second valve, and wherein the at least one gaseous phase of the multi-phase mixture is interchangeable between the measurement tube and the calibration vessel when the second valve is opened.

16. The apparatus of claim 13, wherein the valve system further comprises a third conduit, wherein the third conduit comprises a third valve, wherein the multi-phase mixture in the calibration vessel is fed back to the measurement tube when the third valve is opened, and wherein the third conduit is connected to a flow restriction in the measurement tube.

17. The apparatus of claim 13, wherein the valve system further comprises a fourth conduit, wherein the fourth conduit comprises a fourth valve, and wherein the at least one gaseous phase of the multi-phase mixture in the calibration vessel is released from the calibration vessel when the fourth valve is opened.

18. A method for calibrating an apparatus, for measuring a composition of a multi-phase mixture, the multi-phase mixture comprising at least one liquid phase, at least one gaseous phase, or at least one liquid phase and at least one gaseous phase,
the apparatus comprising:
a measurement tube that forms a conduit configured for receiving a flow of the multi-phase mixture;
a radiation part configured for irradiating the multi-phase mixture in the measurement tube with electromagnetic radiation;
a detector configured for detecting radiation that passes through the multi-phase mixture in the measurement tube;
an analyzer configured for determining the composition of the multi-phase mixture based on the detected radiation and calibration data of the at least one liquid phase and the at least one gaseous phase;
a calibration vessel arranged adjacent to the measurement tube, wherein the radiation part is further configured to irradiate the calibration vessel, wherein the detector is further configured to detect radiation that passes through the calibration vessel, and wherein the calibration vessel is configured to be connected to the measurement tube and to be filled with the multi-phase mixture or respective phases of the multi-phase mixture from the measurement tube; and
a data acquisition part configured for acquiring calibration data from radiation detected by the detector that passes through the calibration vessel when the calibration vessel is filled with the multi-phase mixture or the respective phases of the multi-phase mixture from the measurement tube;
the method comprising:
filling the calibration vessel with the multi-phase mixture or respective phases of the multi-phase mixture from the measurement tube;
irradiating the calibration vessel with electromagnetic radiation from the radiation part;
detecting the radiation that passes through the calibration vessel by the detector; and
acquiring calibration data by the data acquisition part from the radiation detected by the detector.

19. The method of claim 18, further comprising filling the calibration vessel with the at least one gaseous phase and the at least one liquid phase of the multi-phase mixture from the measurement tube, and performing the irradiating and the acquiring for both the at least one liquid phase and the at least one gaseous phase.

20. The method of claim 18, further comprising filling the calibration vessel with the multi-phase mixture from the measurement tube, whereupon stratification of the multi-phase mixture takes place and the at least one liquid phase and the at least one gaseous phase are separated in the calibration vessel, and performing the irradiating and the acquiring after the stratification.

21. The method of claim 20, further comprising separating the at least one liquid phase into an oil phase and a water phase during stratification, and acquiring calibration data for each of the oil phase and the water phase.

22. The method of claim 18, wherein the multi-phase mixture comprises a gas condensate, and wherein a ratio of stable and unstable condensate at atmospheric pressure is determined by the data acquisition part.

23. The apparatus of claim 1 wherein the multi-phase mixture flow comprises a flow of liquid and gaseous hydrocarbons emanating from a well.

24. The apparatus of claim 2 wherein the radiation from the radiation part comprises X-ray radiation, gamma radiation, or x-ray radiation and gamma radiation.
